(19) United States
(12) Patent Application Publication
(10) Pub. No.: US 2015/0073476 A1
(43) Pub. Date: Mar. 12, 2015

(54) SHAPE MEMORY POLYMER COMPOSITIONS

(71) Applicant: Smith & Nephew PLC, London (GB)

(72) Inventors: Malcolm Brown, Otley (GB); Horacio Montes de Oca Balderas, Acomb, York (GB); Michael Andrew Hall, Heslington (GB); Alan William Bull, York (GB); David Franklin Farrar, York (GB); Philip Caton-Rose, Bradford (GB); Philip David Coates, Bradford (GB); Glen Thompson, Bradford (GB); Michael Martyn, Bradford (GB); Ian MacMillan Ward, Leeds (GB); Mark James Bonner, Leeds (GB); Peter John Hine, Leeds (GB); Paul Unwin, Bradford (GB); Robin Anthony Chivers, York (GB); Michael Woodhead, Bradford (GB)

(21) Appl. No.: 14/350,037

(22) PCT Filed: Oct. 5, 2012
(86) PCT No.: PCT/GB2012/052475

§ 371 Date: Apr. 4, 2014

(30) Foreign Application Priority Data
Oct. 5, 2011 (GB) 1117216.0
Oct. 5, 2011 (GB) 1117217.8
Oct. 5, 2011 (GB) 1117218.6
Oct. 5, 2011 (GB) 1117219.4
Oct. 5, 2011 (GB) 1117220.2
Oct. 5, 2011 (GB) 1117222.8
Oct. 5, 2011 (GB) 1117223.6
Oct. 5, 2011 (GB) 1117224.4
Oct. 6, 2011 (GB) 1117214.5
May 29, 2012 (GB) 1209510.5

(51) Int. Cl. A61L 31/14; A61L 31/06; A61L 31/12; A61B 17/04; A61L 31/16

(52) U.S. Cl. CPC A61L 31/148; A61B 17/0401; A61L 31/16; A61L 31/127; A61L 31/141; A61L 31/06; A61B 2017/00871
USPC 606/232; 514/785; 514/8.8; 514/770; 514/546; 424/94.64; 514/16.7; 424/530; 424/93.7

(57) ABSTRACT

The present invention relates to compositions comprising shape memory polymer (SMP) materials and uses thereof. Particularly, although not exclusively, the present invention relates to biocompatible shape memory polymer (SMP) materials and uses thereof in the medical field.

### FIELD OF THE INVENTION

The present invention relates to compositions comprising shape memory polymer (SMP) materials and uses thereof. Particularly, although not exclusively, the present invention relates to biocompatible shape memory polymer (SMP) materials and uses thereof in the medical field. Also included in embodiments of the present invention are medical devices composed wholly or partially of a shape memory polymer material and methods comprising the use of such devices. Embodiments of the present invention comprise resorbable shape memory polymer materials and devices comprising resorbable shape memory polymer materials e.g. tissue anchors and the like.

### BACKGROUND TO THE INVENTION

Shape memory polymers (SMP) are a class of polymers which have the capability of changing their shape upon activation e.g. by application of an increase in temperature. Particularly, SMP have the ability of changing from Shape A to Shape B. Shape A is a temporary shape that is typically obtained by mechanical deformation and subsequent fixation of that deformation (“programming”). Prior to programming, the polymer is formed into its initial permanent shape (Shape B) using conventional techniques such as extruding or injection moulding.

Programming is typically achieved by either heating the sample, deforming, then cooling, or cold drawing or deforming the sample at low temperature. Recovery (to restore to Shape B) has been achieved by heating the device above its transition temperature or reducing the transition temperature to the ambient temperature by allowing a plasticiser to diffuse into the device. The process of programming and recovery of shape memory is represented in FIG. 1.

SMP devices made from polyurethanes have been produced and switching by plasticisation due to absorbed water has been demonstrated. Control of the glass transition temperature (Tg) of these polymers is achieved by modifying the co-polymer composition: Materials today (2007) 10, 4, Polymer (2006)47, 1348, J Mater Chem. 2010 May 14; 20(18): 3356-3366. However, there are no medically approved resorbable versions of these polymers.

Shape memory polymer devices can be used in a variety of applications including, but not limited to, applications in the field of medical devices. An example of their use is in orthopaedic and/or cardiovascular devices. Polymers used for SMP medical devices need to be biocompatible, have mechanical and degradation properties suitable for the specific application in which they are used and change shape at a suitable temperature, and often be resorbable. Examples include sutures, stents, clot removal devices, orthodontics etc. There are difficulties associated with producing such an SMP polymer, particularly a resorbable polymer with suitable mechanical and degradation properties. There are additional difficulties in obtaining SMP materials which are considered suitable for in vivo use.

### SUMMARY OF THE INVENTION

In a first aspect of the present invention, there is provided a composition comprising a biocompatible shape memory polymer material, wherein said composition is for medical use.

Aptly, the shape memory polymer (SMP) material is a non-cross linked semi-crystalline shape memory polymer material. Aptly, the SMP material comprises a polymer selected from poly (D, L)lactide (PDLA), poly(lactic-co-glycolic acid) (PGLA), poly (L, co DL) lactide, poly (L-lactide-co-ε-captolactone) and co-polymers comprising the aforementioned polymers. Aptly, the SMP material comprises an SMP co-polymer.

In one embodiment, the SMP material comprises poly (D,L-lactide). Aptly, the SMP material comprises a poly (D,L-lactide) co-polymer. Aptly, the SMP material comprises poly (D, L-lactide) at a ratio of between about 60-80% by weight L-lactide and between about 20-40% by weight DL-lactide. Aptly, the SMP material comprises poly (D, L-lactide) at a ratio of about 70% L-lactide and about 30% DL-lactide.

Aptly, the composition comprises at least one further component and may comprise at least two further components.

Aptly, the at least one further component is selected from a plasticiser, an inorganic filler, a pharmaceutical agent, a bioactive agent and a magnetic component and combinations thereof. Aptly, the plasticiser is an organic plasticiser e.g. a phthalate derivatives such as dimethyl, diethyl and dibutyl phthalate.

Aptly, the plasticiser is a polyethylene glycol with a molecular weight e.g. from about 200 to 6,000. Details of other suitable plasticisers are provided herein.

Aptly, the plasticizer is a low molecular weight component. Aptly, the plasticiser is selected from the group consisting of DL-lactide, L-lactide, glycolide, ε-Caprolactone, N-methyl-2-pyrrolidinone and a hydrophilic polyol e.g. poly(ethylene) glycol (PEG). In one embodiment, the plasticiser is N-methyl-2-pyrrolidinone (NMP).

Aptly, the composition comprises between about 3% to about 10% w/w of a further component.

In one embodiment, the at least one further component comprises an inorganic filler. The filler may selected from hydroxylapatite, calcium carbonate, calcium phosphate and calcium sulphate. Aptly, the filler is hydroxylapatite. Aptly, the composition comprising about 30% to about 50% w/w of the filler.

Aptly, the SMP material is activated by contact with an aqueous solution having a temperature of approximately 37° C.

Aptly, the at least further component comprises iron oxide.

Aptly, the further component is a bioactive agent. Aptly, the bioactive active is selected from a growth factor, an osteogenic factor, an angiogenic factor, an anti-inflammatory agent and an antimicrobial agent.

In one embodiment, the composition is resorbable.

Aptly, the SMP material is activated by heating above its Tg.

In a further aspect of the present invention, there is provided a device comprising a composition as described herein. Aptly, the device is composed at least in part of a composition as described herein.

Aptly, the device comprises a resorbable SMP material, and the SMP material is capable of being activated by contact with an aqueous solution and further wherein the device is capable of undergoing a shape change upon said contact with an aqueous solution.

Aptly, the device is morphologically stable at a temperature of about 40° C. or lower, when not in contact with an aqueous solution.

Aptly, the device is implantable in a human or animal body.

Aptly, the device is a tissue anchor. Aptly, the device further comprises one or more pores.

Aptly, the device comprises a first component comprising the composition comprising the SMP material and a second component, wherein the SMP material has a first activation temperature and further wherein the SMP material is substantially more deformable at a second activation temperature; and further wherein the second component is deformable by the first component at a temperature equal to or greater than the first activation temperature 1 and which is capable of deforming the first component at a temperature equal to or greater than the second activation temperature.

Aptly, the device comprises an inner layer and an outer layer, wherein the inner layer comprises the first component and the outer layer comprises the second component.

Aptly, the device comprises an inner layer and an outer layer, wherein the inner layer comprises the second component and the outer layer comprises the first component.

In one embodiment, the second component comprises an elastomer.

In one embodiment, the second component comprises an elastomer selected from natural rubber, silicone, polyisoprene, polybutadiene, chloroprene, rubber, butyl rubber, styrene-butadiene rubber, nitrile rubber, ethylene propylene rubber, ethylene propylene diene rubber, polyacrylic rubber, polyether amide, ethylene vinyl acetate and polyurethane.

In one embodiment, wherein the second component comprises a thermoplastic elastomer e.g. a polyurethane, a polyester copolymer, a polyamide copolymer, a styrene-butadiene-styrene block copolymer and a polyolefin copolymer.

In one embodiment, the second component comprises a heat shrink material e.g. a polyolefin.

In one embodiment, the second component comprises a shape memory polymer material having a higher activation temperature than the shape memory polymer material of the first component.

In a further aspect of the present invention, there is provided a method of repairing a soft tissue comprising; placing a device as described herein in bone, passing a flexible member through a soft tissue located adjacent to the bone and tying the flexible member to secure the soft tissue to the body and activating the SMP material such that the device undergoes a radial expansion in at least a section of its length. Aptly, the flexible member is connected to the device prior to placement of the device in the bone.

Aptly, the step of activating the SMP material comprises applying heat to the SMP material. Aptly, the method comprises contacting the SMP material with a heated probe.

Aptly, the method comprises a first step of forming a cavity in the bone and placing the device in the cavity. Aptly, the flexible member is a suture.

In one embodiment, the soft tissue is selected from a tendon, a ligament, a muscle, and cartilage and a combination thereof.

Aptly, the method is for the repair of a rotator cuff.

Aptly, the method is for is for the repair of an anterior cruciate ligament (ACL).

Aptly, the method is for the repair of a glenohumeral instability.

In a further aspect of the present invention, there is provided a method of treating a bone injury comprising implanting a device as described herein into a bone cavity and activating the SMP material. Aptly, the device is an intramedullary nail.

### BRIEF DESCRIPTION OF DRAWINGS

Embodiments of the present invention will now be described hereinafter, by way of example only, with reference to the accompanying drawings in which:

FIG. 1 shows a simplified process of programming and recovery of shape memory material;

FIG. 2 is a graph showing DSC, water uptake and shape recover results of Example 6 in respect of a water activated SMP material;

FIG. 3 is a graph showing the effect of nominally 5% additive on length change of drawn PLDL devices in water at 37° C. as described in Example 7;

FIG. 4 is a graph showing the effect of nominally 10% additive on length change of drawn poly(L-coDL) lactide (PLDL) devices in water at 37° C. as described in Example 7;

FIG. 5 is a graph showing the shape recovery properties of an SMP material comprising PLDL with 4.2% ε-caprolactone and 45.5% large particle hydroxylapatite (HA) as described in Example 8;

FIG. 6 is a graph showing the shape recovery of a drawn cannulated PLDL rod in water at 37° C. of SMP materials as described in Example 9;

FIG. 7 is a graph showing the shape recovery data as described in Example 9 and indicates that the recovery ratio is dependent on the temperature the device is exposed to;

FIG. 8 is a schematic cross sectional view of an embodiment of the present invention which comprises a device having reversible properties. FIGS. 8a and 8b show two constructs with at least partially reversible shape change capability.

FIG. 8a a cross section of a device 300 of embodiments of the invention which comprises an inner component 302 which comprises a SMP material and an outer portion 304 which surrounds the inner portion 302. Aptly, the outer portion is a second material. The device is heated to a temperature T1 which is above the Tg of the SMP material, causing the SMP material to expand radially and stretch the outer portion. Aptly the device 300 is heated again to a temperature which is capable of softening the SMP material such that it is compressed by the second material.

FIG. 8b is a cross section of an alternative embodiment, in which the device 300 comprises an outer ring 306 of an SMP material and an inner portion composed of a second component 308. On heating to a temperature above the Tg of the SMP material, the SMP material contracts, compressing the second material component 308. When heated to a second temperature sufficient to cause the SMP material to soften, the second component expands to increase the cross sectional area of the device.

FIG. 9 is a simplified graph indicating a way in which the relative stiffnesses of the materials may be arranged to change with temperature. Note that by use of the term stiffness here, it is taken to mean the structural stiffness or resistance to deformation of the material, which is a function of both the elastic modulus of the material and its diameter, thickness.

FIG. 10 is a photograph of a reversible shaped device of embodiments of the present invention as described in Example 4;

FIG. 11 is a photograph of the construct described in Example 5;

FIG. 12 is a photograph of a nail construct in an oven as described in Example 5;

FIG. 13 is a graph indicating a fixation temperature profile as described in Example 5;

FIG. 14 is a photograph of the apparatus used to test fixation pull-out as described in Example 5.

FIG. 15 is a graph indicating device pull out test results as described in Example 5;

FIG. 16 is a photograph of the oven used to reverse the shape of a device of an embodiment of the present invention;

FIG. 17 is a graph showing the reversal temperature profile of a device of embodiments of the present invention as described in Example 5; and

FIG. 18 is a photograph of a reversed device and a device which has been pulled out as described in Example 5.

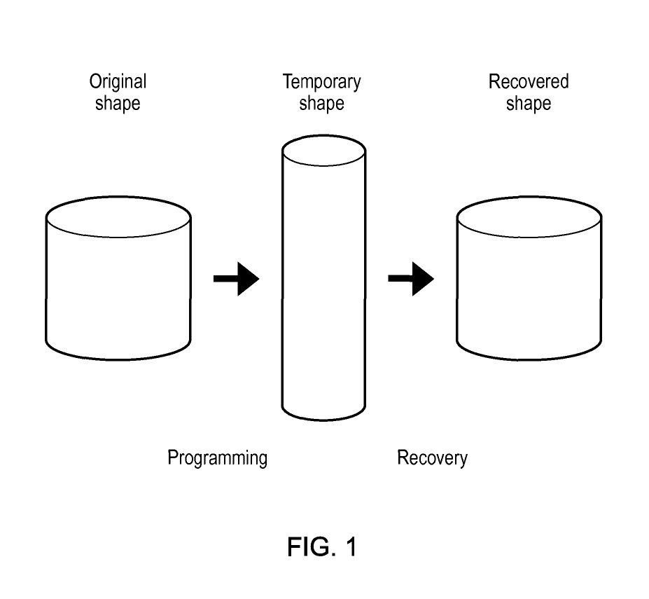

### DETAILED DESCRIPTION OF THE INVENTION

Embodiments of the present invention relate to compositions comprising a shape memory polymer material.

SMP devices made from amorphous polymers such as Poly (D, L-Lactide) (PDLA) can be made and these recover their shape when heated above their glass transition (Tg) temperature ˜40° C. These materials have the advantage that shape recovery can be achieved at low temperatures in vivo minimising the potential for tissue damage. However, amorphous polymers tend to have inferior mechanical properties compared to semi crystalline analogues. In addition, PDLA and other amorphous degradable polymers generally tend to degrade relatively quickly. Such properties can limit the range of device applications in which these SMP can be used.

SMP polymers based on block copolymers are known, such as polyesterurethanes in which the urethane segments act as a hard segments and the ester segment, typically poly(ε-caprolactone) melting (44-55° C.) acts as the switching segment. Shape recovery is achieved by heating the polymer above the melting point of the switching segment. However, there are no medically approved resorbable versions of these copolymers.

Die-drawing is a process that can be used to produce oriented polymers with enhanced mechanical properties. It is a solid state deformation process whereby a polymer billet is heated and drawn through a die of narrower dimensions thus inducing elongation of the billet and orientation of the polymer chains.

In one aspect of the present invention, there is provided a composition comprising an SMP material wherein the SMP has a first shape when a first predetermined condition is satisfied and a second shape when a second predetermined condition is satisfied.

Embodiments of the present invention relate to linear non-cross linked, random, co-polymer semi crystalline shape memory polymers with the composition optimised to allow shape change to occur above their glass transition temperature (Tg), and processes for producing them. Aptly, the process of making a semi-crystalline SMP material involves deforming a semi crystalline or amorphous device at a temperature below its melting point to produce a semi crystalline SMP material that has a different shape and which recovers to produce a semi crystalline device with the original shape or a proportion of its original shape when actuated by heating above the Tg or diffusion of plasticiser to reduce the Tg to below the ambient temperature.

In one embodiment, the SMP material comprises an amorphous SMP polymer.

In one embodiment, the SMP material (comprising amorphous or semi-crystalline polymer) comprises a plasticizer and in addition is activated by application of heat.

In one aspect of the present invention, there is provided a composition comprising a non-cross linked semi-crystalline shape memory polymer (SMP) material. Aptly, the composition is for medical use. Aptly, the composition is for use in the manufacture of a surgical device. Aptly, the SMP material does not comprise a block copolymer. Aptly, the composition is biocompatible. Aptly, the SMP material is biocompatible. Also included in the present invention are devices and products comprising or composed of such compositions.

In one embodiment, the SMP is selected from poly (D, L-lactide) (PDLA). Aptly, the SMP material comprises a non crosslinked PDLA polymer.

Aptly, the SMP material of the composition comprises a filler. Aptly, the filler can be included in smaller quantities as a nucleating agent to accelerate the annealing and produce a more even distribution of crystallinity in the starting device. Aptly, the SMP material comprises between about 0.5% by weight and about 10% by weight of a filler.

Aptly, the filler can also be added in larger quantities to modify the mechanical properties and biocompatibility of the device and function as a nucleating agent. Aptly, the SMP material comprises between about 10% to about 50% by weight.

The initial polymer device can be made using processes such as for example: compression moulding, injection moulding, ram injection moulding or extrusion. Annealing of the device to produce crystallinity can be performed using processes such as: heating in an oven, mould or bath with or without a protective atmosphere.

Deformation of the device to induce shape memory properties can be achieved by processes such as drawing, die drawing or cold forging.

The deformed device may be further modified by processes such as drilling, machining or broaching to produce the desired shape for the SMP device.

Aptly, the composition comprises between 0.5% and 40% w/w, optionally 5% to 35% w/w of an inorganic filler e.g. Hydroxylapatite, calcium phosphate, Calcium sulphate, Calcium carbonate, calcium phosphate or related additives.

In one embodiment, the SMP material comprises Poly(L-co-DL-lactide). Aptly, the SMP material comprises about 70% L-lactide and about 30% DL-Lactide.

Aptly, the semi crystalline polymer is resorbable, e.g. a polyester including random co-polymers containing between 85 to 90% mol/mol Poly(L-lactide) and 15 to 10% of poly(D-lactide) polyglycolide, polycaprolactone, polydanoxanone, or containing 70 to 80% mol/mol Poly(L-lactide) and 20 to 30% of poly(DL-lactide).

Aptly, the composition comprises between about 85 to 90% mol/mol Poly (L-lactide) and between about 15-10% poly(D-lactide). Aptly, the composition comprises between about 85 to 90% Poly (L-lactide) and between about 15-10% polyglycolide.

Aptly, the composition comprises between about 85 to 90% Poly (L-lactide) and between about 15-10% polycaprolactone.

Aptly, the composition comprises between about 85 to 90% Poly (L-lactide) and between about 15-10% polydanoxanone,

Aptly, the composition comprises a pharmaceutical active agent or other bioactive agent e.g. a growth factor, an osteogenic factor, an angiogenic factor, an anti-inflammatory agent, and/or an antimicrobial agent.

Suitable bioactive agents include for example bone morphogenic proteins, antibiotics, anti-inflammatories, angiogenic factors, osteogenic factors, monobutyrin, omental extracts, thrombin, modified proteins, platelet rich plasma/solution, platelet poor plasma/solution, bone marrow aspirate, and any cells sourced from flora or fauna, such as living cells, preserved cells, dormant cells, and dead cells.

It will be appreciated that other bioactive agents known to one of ordinary skill in the art may also be used. Aptly, the active agent is incorporated into the polymeric shape memory material, to be released during the relaxation or degradation of the polymer material. Advantageously, the incorporation of an active agent can act to combat infection at the site of implantation and/or to promote new tissue growth.

Aptly, the SMP material (e.g. an SMP material comprises an amorphous or a semi-crystalline polymer as described herein) comprises a filler. In one embodiment, the filler comprises an inorganic component. Aptly, the filler comprises calcium carbonate, calcium hydrogen carbonate, calcium phosphate, dicalcium phosphate, tricalcium phosphate, magnesium carbonate, sodium carbonate, hydroxyapatite, bone, phosphate glass, silicate glass, sodium phosphate, magnesium phosphate, barium carbonate, barium sulphate, zirconium carbonate, zirconium sulphate, zirconium dioxide, bismuth trioxide, bismuth oxychloride, bismuth carbonate, tungsten oxide and combinations thereof.

Aptly, the composition comprises approximately 0.5% or greater by weight of a filler as described herein. Aptly, the SMP material comprises 0.5%, 1%, 2%, 3%, 5%, 10%, 15%, 20%, 25%, 30%, 35%, 40% or greater by weight of a filler.

Embodiments of the present invention comprising semi crystalline SMP compositions and devices comprising such compositions may have superior properties in comparison to SMP devices made from amorphous polymers such as Poly(D,L-Lactide) (PDLA). This is due to amorphous polymers tending to have inferior mechanical properties compared to those of semi crystalline analogues.

In addition, PDLA and other amorphous degradable polymers tend to degrade more rapidly than their semi crystalline polymers.

Furthermore, SMP polymers based on block copolymers, such as polyesterurethanes, are not medically approved.

Embodiments of the present invention relate to non cross-linked SMP material and devices comprising such material. Compared to cross-linked polymers, these materials are thermoplastic and can be processed by conventional methods such as extrusion, injection moulding etc.

Embodiments of the present invention relate to devices which are adapted to reversibly change shape or dimension. Aptly, the device comprises a shape memory polymer material. Aptly, the SMP material is amorphous. Aptly, the SMP material is semi-crystalline.

In one aspect of the present invention, there is provided a device comprising:
a shape memory component having a activation temperature T1 and which is substantially more deformable at a second activation temperature T2; and
a second component which is deformable by the shape memory component at a temperature equal to or greater than T1 and is capable of deforming the shape memory component at a temperature equal to or greater than T2.

As used herein, the term “deformable” refers to the ability to alter shape and/or dimensions as a result of the application of pressure or stress. Particularly, the term “deformability” as used herein relates to the amount of pressure or force required to cause a shape change. A material which is more deformable than a second material will require less force to be applied to it in order for its shape to be altered.

Thus, embodiments of the present invention are based on dissimilar materials having differing abilities to deform at different temperatures.

Aptly, embodiments of the present invention comprise at least partially reversible shape memory materials and devices. Aptly, one application of the device of the present invention a fixation device such as a screw or anchor, for example a bone screw or tissue anchor which comprises a SMP material. In one embodiment, the device is a nail. Aptly, the device is an intra-medullary nail which is for fixation in bone. In many of these examples it will sometimes be desirable to be able to remove the device.

Under particular circumstances it may be desirable to remove a device formed from a shape memory material from its site of application. In particular, within the medical field, a surgeon may wish to remove a shape memory device, for example, if a site of application has become infected or if it has healed. Due to their mode of action, typical shape memory materials may be difficult to remove.

Reversible shape-memory polymers are known, generally. Those materials tend to display a shape memory effect such that the shape changes on heating and reverses on cooling. However, such polymers are new and have not been used in the body before. Also, they are elastomers with relatively low stiffness so that they may not be suitable for high load bearing applications. It is understood that such materials are not suitable for making a complex device or one which shows radial expansion/contraction as is required for a screw, anchor, tack and the like.

Embodiments of the present invention comprise a multi-component shape-memory construct which is at least partially reversible in shape and/or dimension. Aptly, the reversibility is at least sufficient to allow removal of the shape-memory device when it is used for fixation.

Aptly, the construct comprises:
1) a shape-memory component (Material 1) which has an activation temperature T1 and which is substantially softer at a higher activation temperature T2; and
2) a component (Material 2) that at temperatures greater than or equal to the activation temperature T1 is deformable by the shape-memory component as it undergoes its shape change and is capable of storing sufficient stress for a desired length of time such that at the second activation temperature, T2 (T2>T1) the stress stored in a component comprising Material 2 is capable of compressing/expanding the shape memory component and at least partially reversing the initial shape change.

Aptly, the shape memory component comprises a shape memory polymer.

Thus, embodiments of the present invention comprise a device which comprises a component comprising an SMP material, wherein the device is capable of shape and/or dimension alteration when the SMP material is activated. Aptly, the component is a first component and comprises an SMP material.

Aptly, the shape-memory polymer component (Material 1) may be resorbable or non-resorbable.

In certain applications it may be desired to use an amorphous SMP in the composition.

In one embodiment, the SMP material comprises an amorphous SMP. Alternatively, in one embodiment, the SMP material comprises a semi-crystalline polymer as described herein.

Aptly, the SMP material is resorbable and comprises a resorbable polymer e.g. a polyester for example poly(L-lactide) poly(D,L-lactide), polyglycolide, polycaprolactone, polydioxanone or any blend or copolymer of any of the aforementioned polymers. Aptly, the polymer is a poly(L-lactide) co polymer. Aptly, the SMP material comprises a co-polymer comprising polyglycolide, polycaprolactone and/or polydioxanone.

Aptly, the SMP material is non resorbable and comprises a non-resorbable polymer. Examples of non-resorbable polymers include for example polyurethane, polyacrylate e.g. poly(methyl-methacrylate), poly(butyl methacrylate), poly(ether ether ketone) (PEEK) or any blend or copolymer of the aforementioned polymers.

Other suitable polymers are disclosed herein above.

Aptly, the SMP material comprises filler particles. Aptly, the filler particles are organic or inorganic.

Aptly, the SMP material comprises a bioceramic filler e.g. a calcium phosphate (including for example tricalcium phosphate, hydroxyapatite, brushite and/oroctacalcium phosphate), calcium carbonate, calcium sulphate, and/or a bioglass.

Aptly, the SMP material comprises a pharmaceutical agent e.g. a drug or other bioactive agent e.g. a growth factor, an osteogenic factor, an angiogenic factor, an anti-inflammatory agent, an antibiotic agent and/or an antimicrobial agent or the like. Details of further suitable agents are described herein.

Aptly, the SMP material comprises a plasticiser. Aptly, the plasticiser is capable of modifying the glass transition temperature of the SMP.

Aptly, the plasticiser is a low molecular weight compound. Aptly, the low molecular weight component is selected from the group consisting of DL-lactide, L-lactide, glycolide, ε-Caprolactone, N-methyl-2-pyrolidinone and a hydrophilic polyol e.g. poly(ethylene) glycol (PEG). Aptly the plasticiser is N-methyl-2-pyrolidinone (NMP). Details of other suitable plasticisers are described herein.

Aptly, the SMP material comprises a filler which enables the SMP material to be heated via inductive heating in a magnetic field. Aptly, the filler comprises iron oxide.

The shape memory polymer can be programmed by processes such as die drawing, zone drawing, hydrostatic extrusion, rolling, roll drawing, ram extrusion, compression moulding or any other solid phase deformation process or combination of these that induces molecular orientation in the polymer.

Aptly, the device comprises a second component. The second component comprises a second material. Aptly, the second material comprises a shape-memory polymer material e.g. a SMP material as described above, and having an activation temperature higher than the SMP material of the first component (Material 1).

Aptly, the second component comprises a heat-shrink material e.g. a polyolefin.

Aptly, the second material comprised in the second component comprises an elastomer material e.g. natural rubber, polyisoprene, silicone, polybutadiene, chloroprene rubber, butyl rubber, styrene-butadiene rubber, nitrile rubber, ethylene propylene rubber, ethylene propylene diene rubber, polyacrylic rubber, polyether amide, ethylene vinyl acetate and/or polyurethane.

Aptly, the second material comprised in the second component comprises a thermoplastic elastomer e.g. a polyurethane, a polyester copolymer, a polyamide copolymer, a styrene-butadiene-styrene block copolymer and/or a polyolefin copolymer.

Aptly, the device can be activated by direct heat e.g. using a thermal probe, heated liquid, and/or body heat and the like. Aptly, the device can be activated by indirect heat e.g. by inductive heating in a magnetic field; diffusion of a solvent (e.g. water) to lower the transition temperature and/or light and the like.

In one embodiment, the device is for non-medical use. Aptly, the device is used in a non-medical application e.g. such as engineering fixation devices and fasteners.

Aptly, the device is a fastener that allows for rapid dis-assembly of a device/product at the end of its life e.g. by simply heating. This is a benefit in recycling of products.

Embodiments of the present invention which comprise a first component comprising an SMP material and a second component comprising a second material may provide a device which allows for easy and complete removal of the device from a location. For a medical device this may be necessary if for example the device is infected and needs to be removed or if it is desired to remove the device once the body has healed, for example. The device may also allow for rapid tamper fastening or closure.

Embodiments of the present invention provide advantages over other reversible shape-memory materials since the devices of the present invention can be constructed from readily available polymers and materials. For medical devices this means materials with a history of use in the body can be used. Also, many currently known reversible SMPs do not have high enough mechanical properties to allow for strong fixation, if that is the desired use.

Embodiments of the present invention relate to compositions and devices composed thereof, either wholly or in part, which include a SMP material which is activated by an aqueous solution that is at around body temperature (e.g. approximately 37° C.). In an aspect of the present invention, there is provided a composition comprising a SMP material, wherein the SMP material is activated by an aqueous solution that is at around body temperature (e.g. approximately 37° C.). Aptly, the SMP material comprises a plasticiser.

SMP devices made from polyurethanes have been produced and switching by plasticisation due to absorbed water has been demonstrated, control of the Tg of these polymers is achieved by modifying the co-polymer composition: Materials today (2007) 10, 4, Polymer (2006)47, 1348, J Mater Chem. 2010 May 14; 20(18): 3356-3366.

However, there are no medically approved resorbable versions of these polymers.

Embodiments of the present invention relate to a device comprising an SMP material aptly a resorbable medical polymer. Aptly, the device is morphologically stable at ambient temperature or up to ˜40° C., so it is stable within the range it may experience during transportation and storage.

Aptly, the device can have the aspect of a conventional medical screw or anchor and can be re-positioned and adjusted as required during surgery. The device will typically change shape post implantation due to plasticisation caused by absorption of water.

Aptly, the time taken for the shape to change can be controlled to occur over a time frame of minutes or hours as required for a particular application by controlling the formulation and aspect of the device. For some polymers, formulation with a low molecular weight biocompatible compound to plasticise the polymer is required. Aptly, the SMP material comprises a low molecular weight biocompatible compound which reduces the glass transition temperature (Tg) of the polymer so that the further plasticisation achieved by water absorption is sufficient to allow shape recovery at 37° C. or lower. By adjusting the formulation, both the rate of shape recovery and maximum stable storage temperature can be controlled.

Aptly, the device can be produced such that it exhibits a different degree of shape recovery at different temperatures. Aptly, the device is adapted to encourage bone in growth. Aptly, the device comprises one or more pores. Aptly, the device comprises a SMP material which comprises an osteoconductive filler. The osteoconductive filler comprises a bioceramic.

In one aspect of the present invention, there is provided a device comprising a resorbable shape memory polymer (SMP) material, wherein the SMP material is capable of being activated by contact with an aqueous solution, and further wherein the device is capable of undergoing a shape change upon said contact with an aqueous solution.

Aptly, the device is morphologically stable at a temperature of about 40° C. or lower.

Aptly, the SMP material comprises a plasticiser. Plasticisers or mixtures thereof suitable for use in the present invention may be selected from a variety of materials including organic plasticisers and those like water that do not-contain organic compounds.

Aptly, the plasticiser is selected from DL-lactide, L-lactide, glycolide, ε-Caprolactone, N-methyl-2-pyrrolidinone and a hydrophilic polyol e.g. poly(ethylene) glycol (PEG).

Plasticisers or mixtures thereof suitable for use in the present invention may be selected from a variety of materials including organic plasticisers and those like water that do not-contain organic compounds.

Aptly, the plasticiser is an organic plasticiser e.g. a phthalate derivatives such as dimethyl, diethyl and dibutyl phthalate; a polyethylene glycol with a molecular weight e.g. from about 200 to 6,000, glycerol, glycols e.g. polypropylene, propylene, polyethylene and ethylene glycol; citrate esters e.g. tributyl, triethyl, triacetyl, acetyl triethyl, and acetyl tributyl citrates, surfactants e.g. sodium dodecyl sulfate and polyoxymethylene (20) sorbitan and polyoxyethylene (20) sorbitan monooleate, organic solvents such as 1,4-dioxane, chloroform, ethanol and isopropyl alcohol and their mixtures with other solvents such as acetone and ethyl acetate, organic acids such as acetic acid and lactic acids and their alkyl esters, bulk sweeteners such as sorbitol, mannitol, xylitol and lycasin, fats/oils such as vegetable oil, seed oil and castor oil, acetylated monoglyceride, triacetin, sucrose esters, or mixtures thereof.

Aptly, the plasticiser is selected from a citrate ester; a polyethylene glycol and dioxane.

Aptly, the SMP material comprises a filler. Aptly, the device may contain between 0.5% and 40% w/w, e.g. 5% to 35% w/w inorganic filler such as: Hydroxylapatite, Tricalcium phosphate, Calcium sulphate, Calcium carbonate, dicalcium phosphate or similar.

The semi crystalline polymer can be resorbable such as: a polyester including random co-polymers containing between 85 to 90% mol/mol Poly(L-lactide) and 15 to 10% of poly(D-lactide) polyglycolide, polycaprolactone, polydanoxanone or containing 70 to 80% mol/mol Poly(L-lactide) and 20 to 30% of poly(DL-lactide).

The polymer can also include drugs or other bioactive agents such as a growth factor, osteogenic factors angiogenic factor, anti-inflammatory agent, antimicrobial etc.

In one embodiment, the SMP material comprises a HA filled Poly(L-co-DL-lactide) (ratio:80:20) and a ε-Caprolactone.

Embodiments of the present invention provide body temperature shape memory devices which are superior to conventional medical fixation devices as they will provide equivalent fixation immediately on application then, after the surgery is completed automatically change shape to increase fixation. These devices may also offer advantages over other shape memory devices as they do not need to be activated with heat or other means to achieve fixation and can be adjusted if required during surgery as they do not rely on their shape memory properties to achieve initial fixation.

As described herein, the present invention includes medical devices composed of in whole or in part of a shape memory polymer (SMP) material. In one aspect of the present invention, there is provided a device comprising a composition as described herein. Aptly, the device is for medical use. Aptly, the device is for surgical use. In one embodiment, the device is a fixation device for example a tissue anchor or the like. In one embodiment, the fixation device is a suture anchor. Aptly, the device is a device adapted to be inserted into a cavity within the body of a patient.

Aptly, the device is selected from a group consisting of pins, rods, nails, screws, plates, anchors and wedges.

In one embodiment, the device is for medical use. In one embodiment, the device is a surgical device. Aptly, the device is an implant e.g. tissue anchor. Aptly, the device is a suture anchor, a. In one embodiment, the device is for use in the fixation of a glenohumeral instability in a patient. Aptly, the device is a nail, a wedge, an anchor or the like. In one embodiment, the nail is an intramedullary nail.

Aptly, the device is an implantable device and may be applied with respect to bone, soft tissue and other elements in a surgical site. The term “surgical site” as used herein may include any portion of a patient to which operating personnel may have access during surgery. The term “patient” may refer to human and non-human patients.

The initial polymer device can be made using processes such as: compression moulding, injection moulding, ram injection moulding or extrusion and may be further modified by processes such as drilling, machining or broaching to produce the desired shape.

Deformation of the device to induce shape memory properties can be achieved by processes such as drawing, die drawing or forging. The deformed device may be further modified by processes such as drilling, machining or broaching to produce the desired shape for the SMP device. Other methods for example cold forging and overmoulding may be used to shape the SMP material devices. These processes and other processes and devices are subject of our co-pending patent applications which have a common priority to the present invention and the contents of which are incorporated herein by reference.

The plasticiser and/or other filler and/or pharmaceutical agent can be added to a polymer composition prior to programming of the polymer to impact shape memory properties to the polymer and thus form the SMP material.

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### EXAMPLES

### Example 1

### Production of Semi-Crystalline SMP Material

Poly(L-co-DL-lactide), 70% L-lactide, 30% DL-Lactide, PURASORB PLDL 7038 (PURAC Biochem B.V.) was compression moulded to produce a 30 mm diameter billet. A 30 mm diameter billet mould was pre-heated to 160° C., granules added and the billet moulded under 10 tons of pressure applied using a hydraulic press. The billet was then annealed at 105° C. for 12 days in a dry nitrogen atmosphere to allow crystallisation to occur, samples were then removed for DSC analysis.

The semi-crystalline billet was then drawn through a 15 mm diameter die at 75° C. at a rate of 30 mm min⁻¹ to produce a rod with shape memory properties.

Drawn rods were cut into lengths and sampled at various points for DSC analysis. The rods were shape recovered in hot water (˜90° C.) for 10 minutes, then re-sampled for differential scanning calorimetry (DSC) analysis.

DSC was used to determine the crystallinity of the rods pre and post drawing and post shape recovery by measuring the area (ΔH Jg⁻¹) of the melting peak at 120° C.: Samples were heated at 10° C. min-1, from 20 to 200° C. The results shown in Table 1 below indicate that the rods retained crystallinity through die drawing and shape recovery.

TABLE 1

| | Billet 23 | ΔH (J/g) | |
|---|---|---|---|
| Process stage | sample position | Inside | Outside |
| Pre-draw billet | Top | | 7.241 |
| | Bottom | 4.017 | 1.132 |
| Post-Draw rod | Top | 1.141 | 1.618 |
| | Bottom | 0.888 | 0.497 |
| Post shape recovery rod. | Top | 0.952 | 2.272 |
| | Bottom | 0.710 | 0.127 |

The crystallinity of the billets pre-drawing was variable, in all of the samples examined, some degree of crystallinity was maintained through drawing and post shape recovery.

### Example 2

Poly(L-co-DL-lactide), 70% L-lactide, 30% DL-Lactide, (molar ratio) PURASORB PLDL 7038 (PURAC Biochem B.V., Holland) was compounded with Hydroxylapatite Powder P220 S (Plasma Biotal Limited) to produce granules containing 35% w/w hydroxylapatite. Granules were compression moulded to produce a 30 mm diameter billets. A 30 mm diameter billet mould was pre-heated to 160° C., granules added and the billet moulded under 10 tons of pressure applied using a hydraulic press. The billet was then annealed at 105° C. for 9 days in a dry nitrogen atmosphere to allow crystallisation to occur, samples were then removed for DSC analysis.

The semi-crystalline billet was then drawn through a 15 mm diameter die at 80° C. at a rate 30 mm min⁻¹ to produce a rod with shape memory properties.

Drawn rods were cut into lengths and sampled at various points for DSC analysis. The rods were shape recovered in hot water (˜90° C.) for 10 minutes, then re-sampled for DSC analysis.

DSC was used to determine the crystallinity of the rods pre and post drawing and post shape recovery by measuring the area (ΔH Jg⁻¹) of the melting peak at 120° C.: Samples were heated at 10° C. min-1, from 20 to 200° C. The results shown below in Table 2 indicate that the rods retained crystallinity through die drawing and shape recovery.

Table 2
Crystallinity of annealed, drawn and shape recovered billet.

| | Billet | ΔH |
|---|---|---|
| Process stage | ET258-8800-3-5 | (J/g) |
| Pre-draw billet | Tip | 7.84 |
| | Bottom | 6.93 |
| | Mean | 7.39 |
| Post-Draw rod | 15 mm/min | 4.37 |
| | 20 mm/min | 4.08 |
| | 30 mm/min | 4.41 |
| | Mean | 4.29 |
| Post shape recovery rod | 15 mm/min | 3.37 |
| | 20 mm/min | 3.22 |
| | 30 mm/min | 2.94 |
| | Mean | 3.18 |

### Example 3

30 mm diameter billets moulded from Poly(L-co-DL-lactide) which comprised 70% L-lactide, 30% DL-Lactide, (molar ratio) PURASORB PLDL 7038 (PURAC Biochem B.V.) and 35% w/w CaCO₃ samples were die drawn through 10, 12 and 15 mm dies at a range of draw rates to produce shape memory rods with a range of draw ratios. DSC was used to determine the crystallinity of the rods pre and post drawing and post shape recovery by measuring the area (ΔH Jg⁻¹) of the melting peak at 120° C.: Samples were heated at 10° C. min-1, from 20 to 200° C. The draw ratios, draw rates and AH are shown in Table 3, showing that drawing at ratios above 4 produced a semi crystalline shape memory polymer.

TABLE 3
Draw induced crystallinity,
effect of draw ratio and rate on ΔH

| Draw | | ΔH |
|---|---|---|
| ratio | rate | (J/g) |
| 4 | 55 | 0.0 |
| 5.7 | 50 | 0.8 |
| 9 | 10 | 2.0 |
| | 30 | 1.7 |
| | 40 | 2.5 |
| | | 3.6 |

Shape memory rods were placed in water at 90° C. to allow shape recovery to occur and the shape recovery ratio calculated (Table 4):

Shape recovery of semi crystalline shape memory polymers.

$$Recovery ratio = \frac{Post recovery cross sectional area = {( post recovery diameter )}^{2}}{Post draw cross sectional area = {( post draw diameter )}^{2}}$$

TABLE 4

| Draw | Draw rate | Diameter (mm) | | Recovery |
|---|---|---|---|---|
| Ratio | (mm/min) | Post draw | Post recovery | ratio |
| 4.0 | 55 | 15 | 28.5 | 3.61 |
| 5.7 | 50 | 12 | 24.5 | 4.17 |
| 9.0 | 10 | 10 | 15.0 | 2.26 |
| | 30 | 10 | 17.1 | 2.93 |
| | 40 | 10 | 18.0 | 3.25 |

The initial polymer device (pre-programming) can be made using processes such as: compression moulding, injection moulding, ram injection moulding or extrusion. Annealing of the device to produce crystallinity can be performed using processes such as: heating in an oven, mould or bath with or without a protective atmosphere. The polymer device was heated above the Tg of the polymer but below the melting point of the polymer.

### Example 4

### Preparation of Reversible Shape Memory Polymer Material Devices

Poly(L-lactide-co-DL-lactide) 70:30 with IV=3.8 (Purasorb PLDL7038,Purac Biomaterials) was blended with 5% caprolactone as a plasticiser. The polymer was die-drawn to a draw ratio of 4:1 with a final diameter of 8 mm.

Three constructs were made prepared in which a piece of the programmed shape-memory polymer described above was covered with an outer layer of:
a) natural rubber modelling balloon approx. 0.2 mm wall thickness
b) Silicone lab tubing 8 mm internal diameter, 1.5 mm wall thickness
c) Heat-shrink polyolefin tubing RNF-3000 12/4 supplied by TYCO/ISRayfast

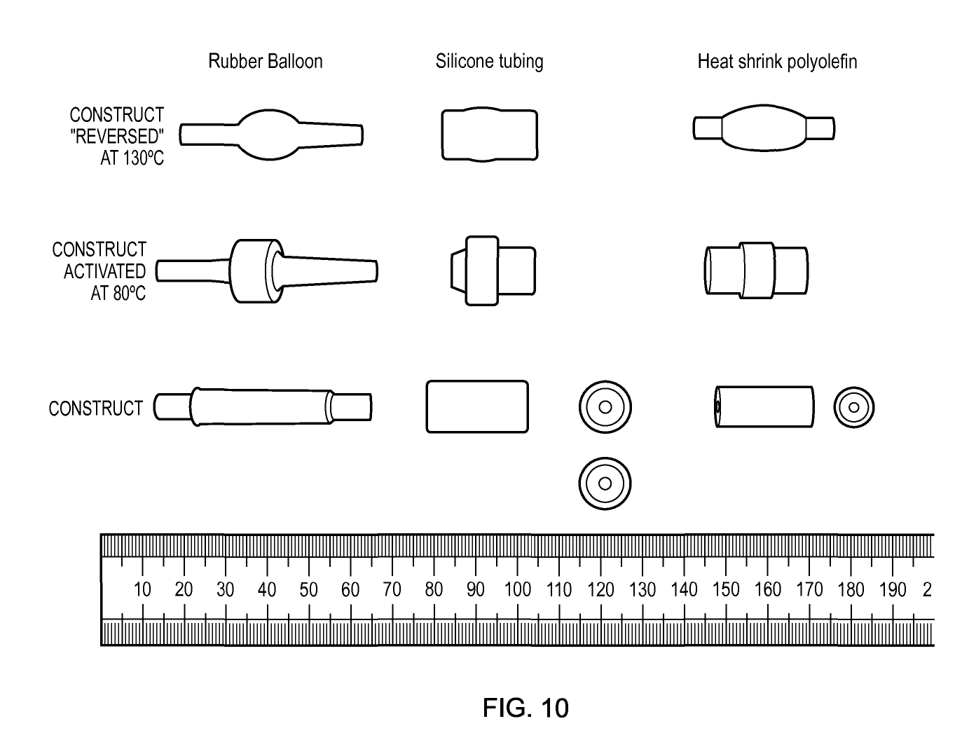

The constructs were activated by heating to a temperature of 80° C., at which temperature the inner PLDL7038/CL SMP component had expanded. In order to partially reverse the shape change, the constructs were then heated to 130° C., at which temperature the inner polymer had softened considerably and was deformed back towards its original diameter by the stress applied by the outer rubber/silicone/polyolefin layer (see FIG. 10).

### Example 5

### Nail Fixation Testing

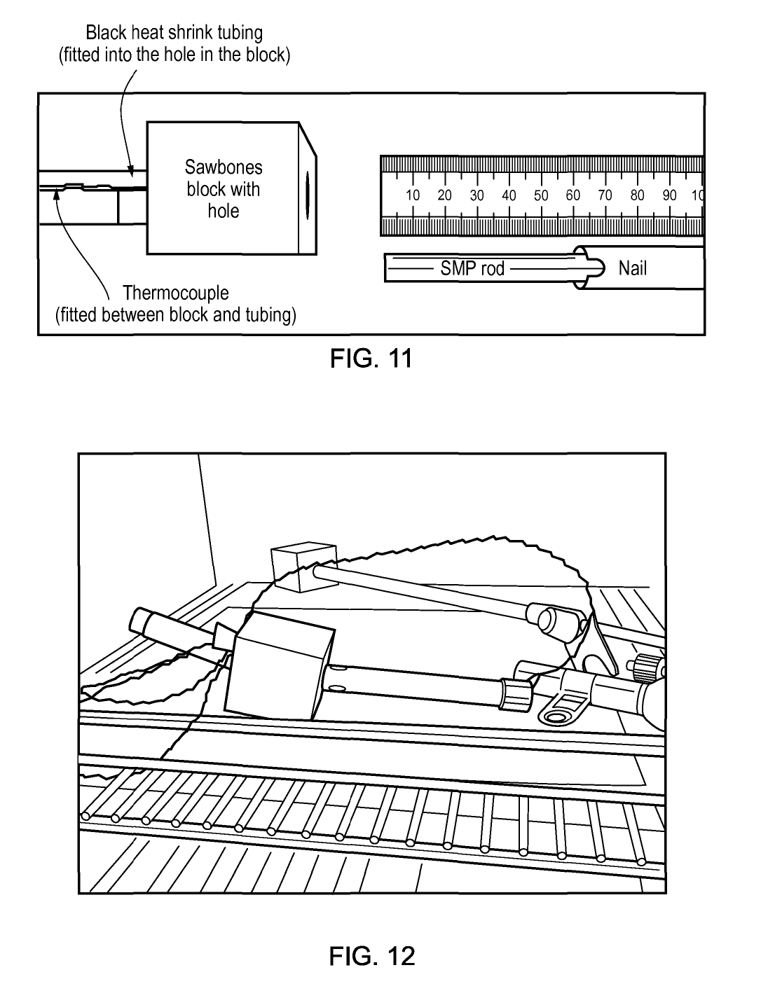

An intra-medullary nail (Smith & Nephew, Inc.) was selected that had an 8 mm internal diameter and the 8 mm PLDL/caprolactone cannulated rod was a perfect snug fit. The nail construct comprising the SMP rod, heat shrink tubing, “Sawbones®” test block (20 pcf, supplied by Sawbones AB, Sweden, with 14 mm drilled hole), nail and the thermocouples is shown pre-assembly in FIG. 11. The thermocouples that were placed inside the cannulated rod and at the Sawbones®/heatshrink interface were monitored during fixation and reversal using a Comark temperature logger model-N2104.

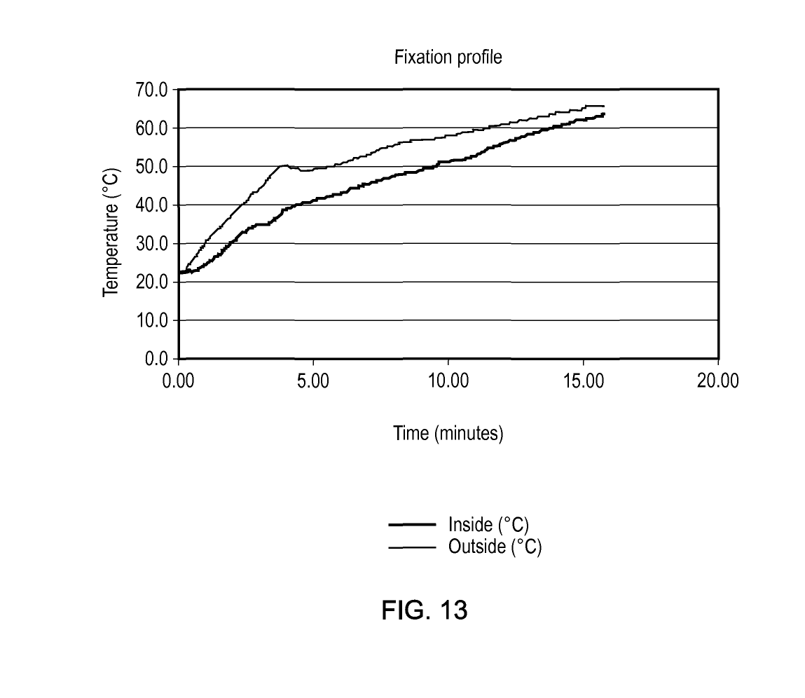

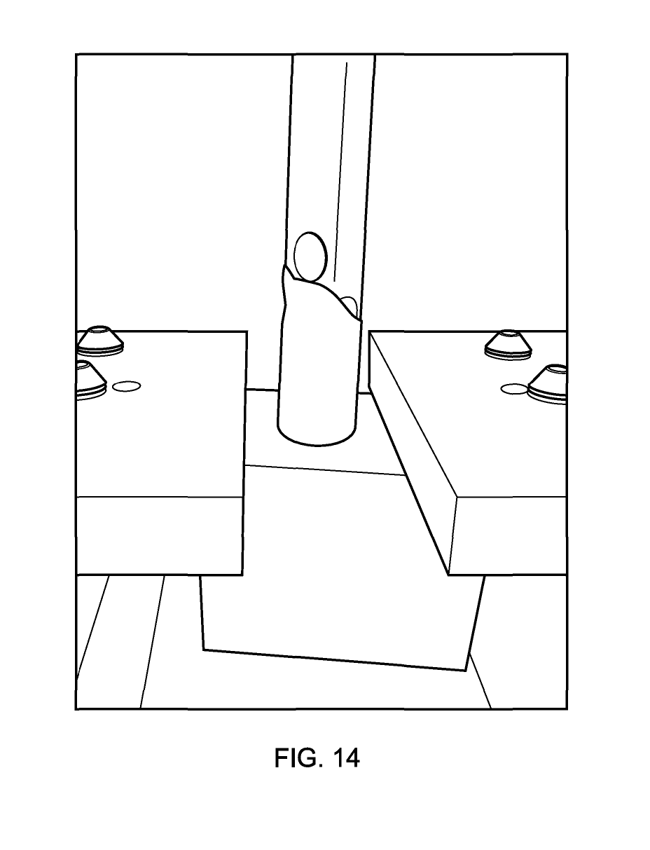

The preassembled device and Sawbones® construct was placed in an oven (Gallenkamp) set at 80° C. Monitoring of the construct and observation of the temperature indicated that fixation had been achieved after approximately 16 minutes when the temperature had reached approximately 65° C. The fixation construct setup and temperature profile measured are shown in FIG. 13. Two fixed nail samples were prepared. One was prepared for pull-out testing and the other for demonstration of reversibility.

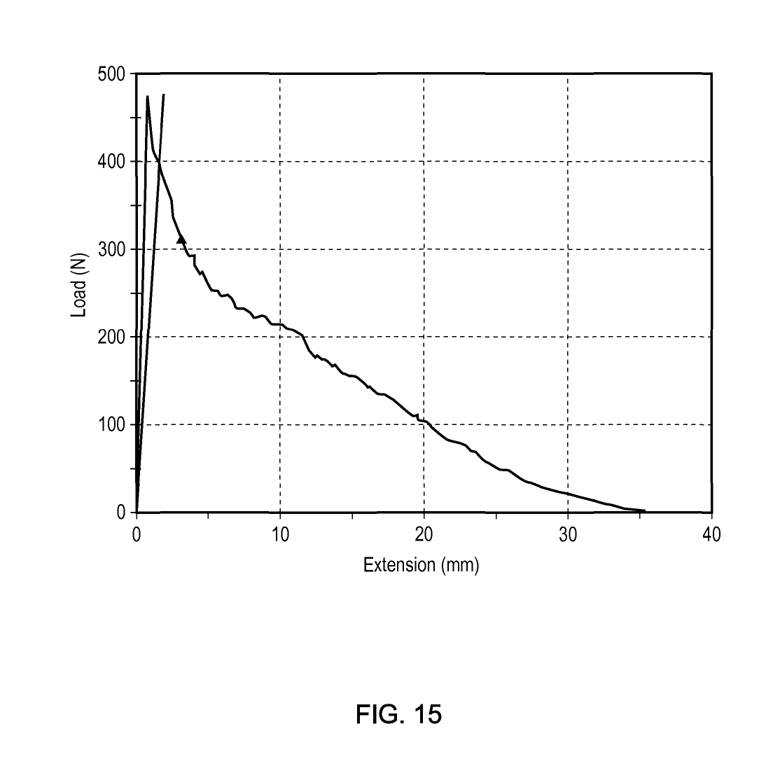

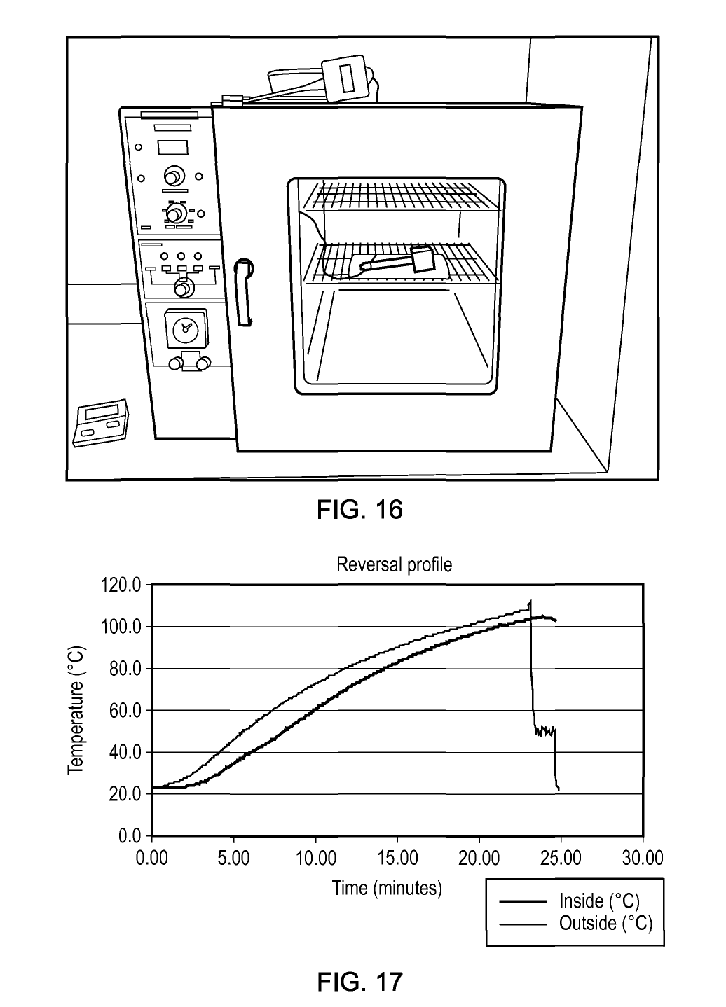

The pull-out strength of the nail construct in the Sawbones was measured using an Instron 5566 universal testing machine at a rate of 10 mm/min. The pull-out force was measured as 480N (FIG. 15). Failure was noted between the heat shrink and the Sawbones interface. One of the fixed devices was reversed by placing it in the oven set at 130° C. The setup and reversal temperature profile are shown in FIG. 17.

The device released from the hole after 24 minutes heating. At this time the temperature of the sleeve approached 110° C.

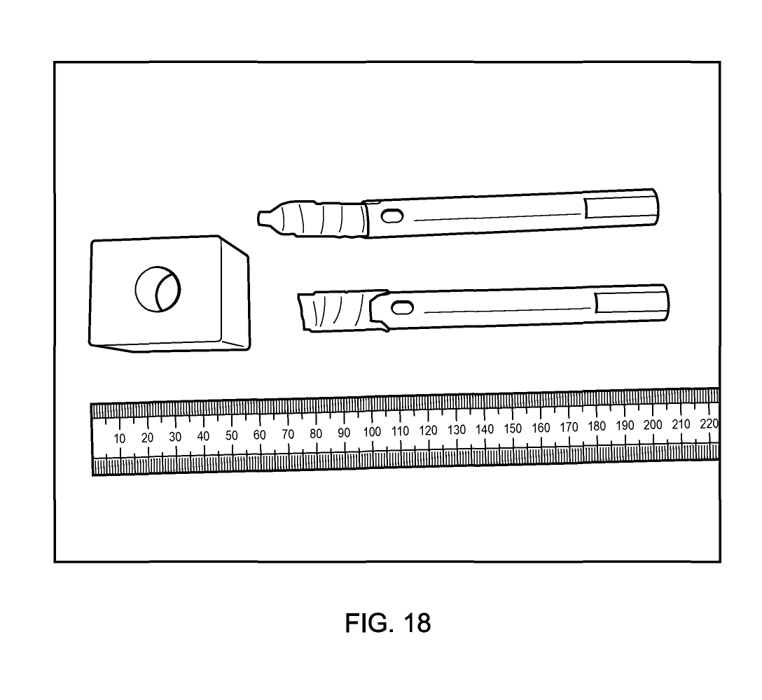

The reversed and pulled out nails are shown in FIG. 18. The reversed nail's rounded nose slipped out of the Sawbones hole after reversal.

### Example 6

### SMP Material Composition which is Activated by Water at Body Temperature

Granules of Poly(L-Lactide-co-glycolide) 85:15 (Purac Biochem B.V. lot 0408001846) were compression moulded at 200° C. under 50 kN pressure to produce polymer sheets approx. 0.25 mm thick. The moulded polymer sheets were then cut to produce devices.

The devices were placed in an oven at 80° C. Once they had warmed up they were removed from the oven and drawn by hand. One specimen of each type was returned to the oven at 80° C. and shape recovery observed, confirming the device had SMP properties.

Devices were weighed and a 50 mm length was marked on the drawn section and its recorded. The devices were then placed in water at 37° C. and removed and re-measured periodically. The weight changes were used to calculate the water uptake and distance between the marks to measure the change in length.

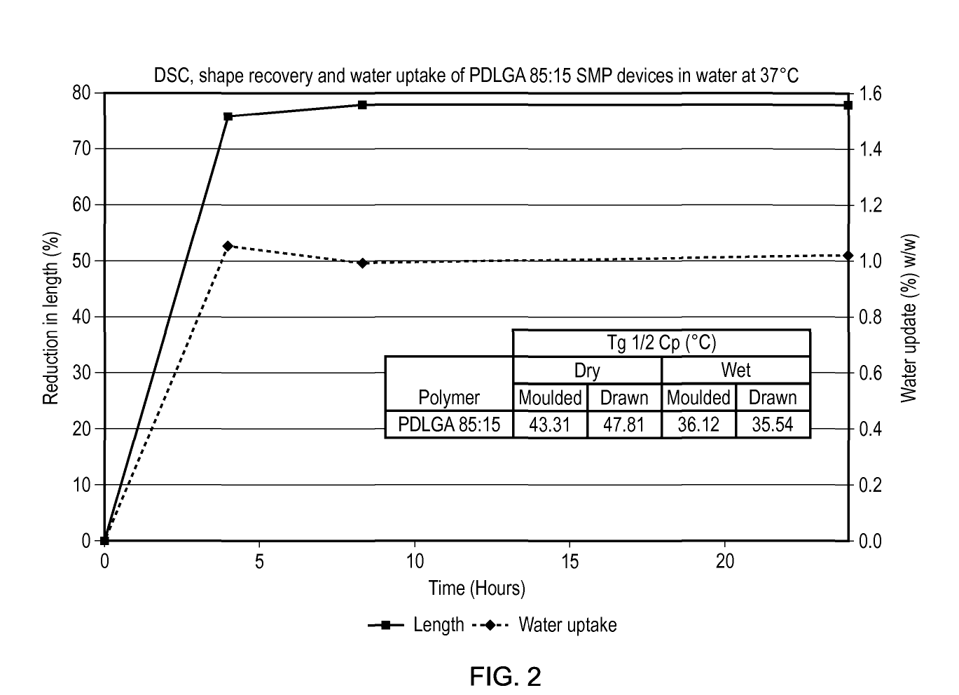

DSC was used to measure the Tg (½ Cp) of the devices: One heat cycle between 10 and 200° C. with a heating rate 10° C. min-1 was used. The Tg of wet and dry moulded and drawn devices were measured prior to shape recovery. The DSC, water uptake and shape recovery results are shown in FIG. 2.

### Example 7

Plasticisation of Poly(L-co-DL-lactide) 70:30 with a small molecule to produce a body temperature water activated SMP.

Poly(L-co-DL-lactide) 70:30 (PURASORB PLDL 7038, PURAC biochem by.) granules were tumble blended to give mixtures nominally containing 5 or 10% w/w of a range of low molecular weight biocompatible compounds: DL-Lactide (PURAC biochem by), L-Lactide (PURAC biochem bv), Glycolide (PURAC biochem by), ε-Caprolactone (Fluka 21510), N-Methyl-2-Pyrolidinone (NMP) (Aldrich 32863-4). The mixtures were then compounded using a Haake MiniLab twin-screw extruder at 50 rpm, at temperatures between 190 and 210° C., adjusted as necessary to control the viscosity of the extrudate for each formulation. The extrudate was pelletised and additive content confirmed by Gas Chromatography—Flame Ionisation Detection (GC-FID).

Granules of each formulation were compression moulded at 200° C. under 50 kN pressure to produce polymer sheets approx. 0.25 mm thick. The moulded polymer sheets were then cut to produce devices.

The devices were placed in an oven at 80° C. Once they had warmed up they were removed from the oven and drawn by hand. One specimen of each type was returned to the oven at 80° C. and shape recovery observed, confirming the device had SMP properties.

DSC was used to measure the Tg (½ Cp) of the devices: One heat cycle between 10 and 200° C. with a heating rate 10° C. min-1 was used. The Tg of wet and dry moulded and drawn devices were measured prior to shape recovery.

A 50 mm length was marked on the drawn section of each device. The devices were then placed in water at 37° C. and periodically removed for re-measurement. The changes in the distance between the marks were used to determine the change in length.

The composition of the devices and DSC results for the formulations is shown in Table 5 below:

TABLE 5

| Plasticiser | | Dry Tg (½ Cp) | | Wet Tg (½ Cp) | |
|---|---|---|---|---|---|
| Type | % (w/w) | Moulded | Drawn | Moulded | Drawn |
| None | 0.0 | 52.7 | 59.8 | 50.1 | 48.4 |
| ε-Caprolactone | 3.9 | 47.3 | 46.2 | 38.8 | 36.0 |
| ε-Caprolactone | 8.3 | 36.9 | 39.6 | 30.4 | 37.3 |
| DL-Lactide | 3.3 | 51.3 | 55.9 | 41.0 | 54.2 |
| DL-Lactide | 7.2 | 41.4 | 41.2 | 36.6 | 40.5 |
| Glycolide | 2.4 | 50.7 | 48.8 | 46.7 | 45.8 |
| Glycolide | 7.0 | 41.8 | 43.7 | 33.4 | 35.8 |
| L-Lactide | 3.4 | 47.5 | 53.4 | 42.6 | 56.0 |
| L-Lactide | 7.2 | 41.0 | 41.0 | 37.6 | 36.0 |
| NMP | 6.3 | 39.3 | 41.3 | 32.4 | 32.5 |

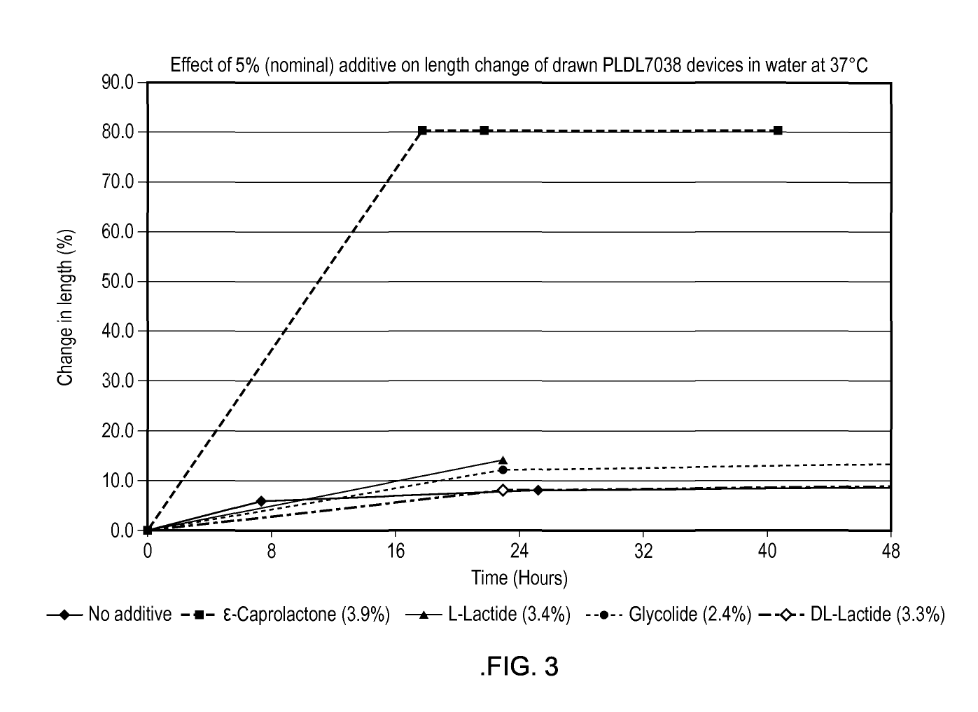

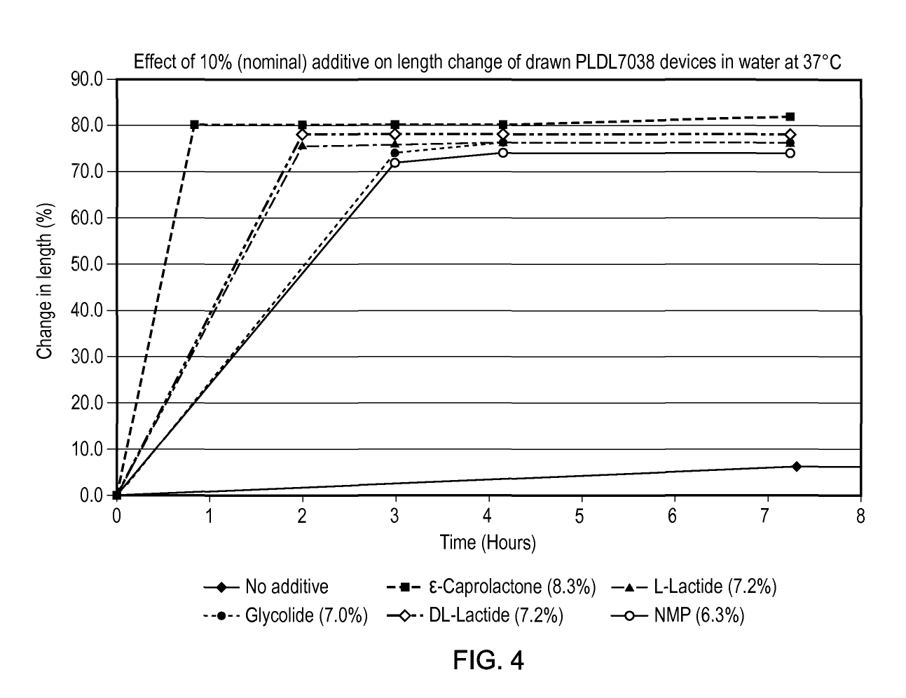

Shape recovery of these devices in water at 37° C. was shown to occur with formulations that had wet Tg close to or below 37° C. (See FIGS. 3 and 4)

### Example 8

Body temperature water activated SMP with filler.

200 g of Poly(L-co-DL-lactide) 70:30 (PURASORB PLDL 7038, PURAC biochem by.) were tumble blended with 10.52 g ε-Caprolactone (Fluka 21510) for 2 days. 165.4 g of Hydroxylapatite (HA) sieve fraction 180 to 355 μm, P216 S 355<180 XRD 3303 (Plasma Biotal ltd) were then added and tumble blended for 16 hours. The mixture was then compounded using a Prism TSE 16TC extruder to produce homogenous granules.

The granules were compression moulded at 165° C. to produce a 30 mm diameter billet suitable for die drawing. The moulded billet was die drawn through a 15 mm die at 75° C. at 30 mm/min, yielding a drawn porous SMP rod 15.46 mm in diameter, draw ratio 3.77. A portion of the rod was placed in hot water (˜90° C.) and shape recovery observed, confirming the device had SMP properties. The presence of the large filler particles caused pores to open in the rod during drawing. The porosity of the drawn rod was 41.7% by volume, calculated from the weight and volume of a small section and the density of the component materials. Analysis of the rod yielded HA content of the formulation: 45.5% w/w (by ashing) and ε-Caprolactone content of the organic fraction of the formulation: 4.2% w/w (by GC-FID).

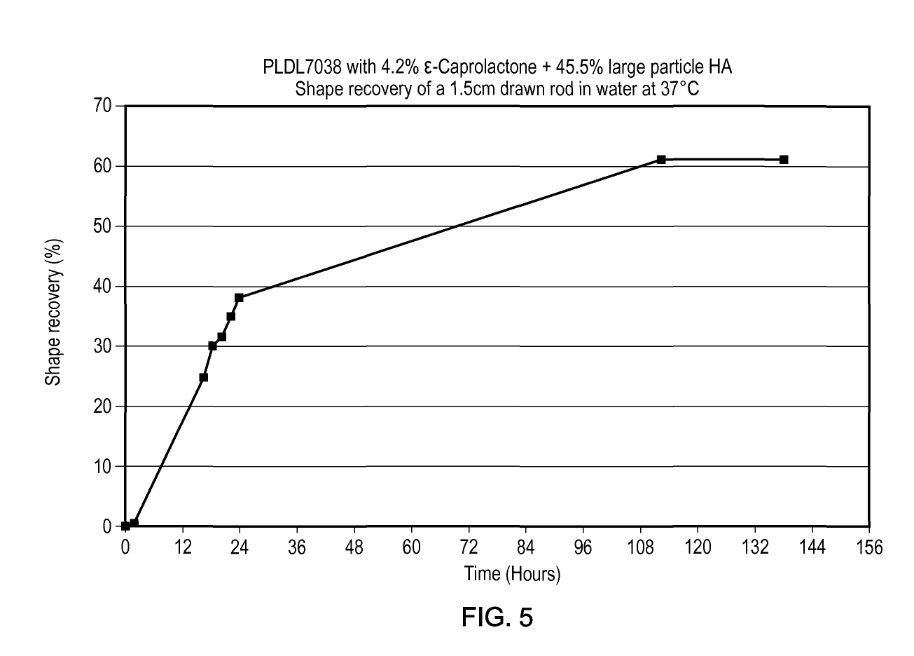

A 1.530 cm length of the rod was placed in water at 37° C. and periodically removed for re-measurement. The shape recovery of the device is shown in FIG. 5.

### Example 9

Plasticisation of HA filled Poly(L-co-DL-lactide) 80:20 with a ε-Caprolactone to produce a body temperature water activated SMP.

Poly(L-co-DL-lactide) 80:20 (PURASORB PLDL 8038 lot 0807000756 PURAC biochem by) were compounded with Hydroxylapatite Powder (HA) (P220 S Batch:-P220 S XRD Ident:-3703 Plasma Biotal Limited) and ε-Caprolactone (Fluka 21510) to produce pellets of two formulations, both nominally containing 5% w/w HA one formulation also nominally containing 5% w/w (of the organic fraction) ε-Caprolactone.

Pellets of these materials were melt extruded to produce solid billets of diameter 14 mm. These billets were given a 3.5 cm cannulation by drilling then die drawn at 65 to 72° C., 30 mm min⁻¹ through a 7 mm die with a 3 mm hex mandrel fitted.

The rods were then analysed to determine the composition; the HA content of by ashing, the ε-Caprolactone content by GC-FID, the molecular weight by GPC (Table 6).

TABLE 6

| | HA (% w/w of | | Caprolactone (% w/w |
|---|---|---|---|
| Discription | Formulation) | Mn | of organics) |
| PLDL 8038 + | 4.60 | 223093 | — |
| 5% w/w HA | | | |
| PLDL 8038 + | 4.90 | 226293 | 4.08 |
| 5% w/w HA & | | | |
| Caprolactone | | | |

1 cm lengths were cut from each of the formulations and measured accurately; the samples were then placed in water at 37° C. Periodically the samples were removed from the water, re-measured then replaced in the water. The sample length data obtained was used to calculate the percentage shape recovery:

$$Shape recovery \% = \frac{Original length - New length}{Original length - ( Original length / Nominal draw ratio )} \times 100$$

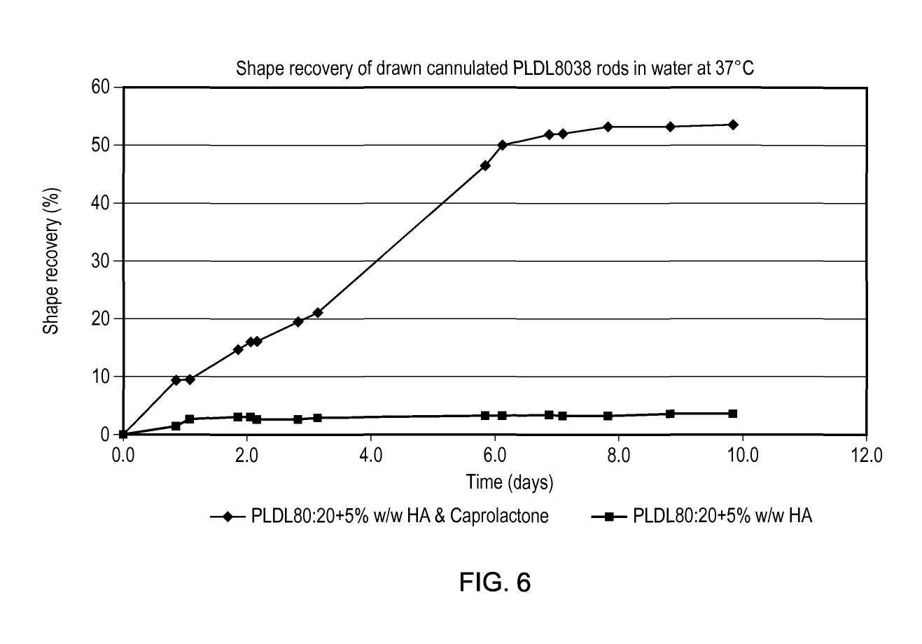

The results (FIG. 6) show that the formulation plasticised with caprolactone showed significant shape recovery in water at 37° C. and the un-plasticised formulation did not.

### Example 10

Controlling the temperature recovery response of devices by manipulating the draw temperature.

Four 14 mm diameter solid billets were moulded from pellets of Poly(L-co-DL-lactide) 80:20 (PURASORB PLDL 8038 PURAC biochem bv) containing 5.35% caprolactone (Fluka 21510). These billets were given a 3.5 cm cannulation by drilling and a series of SMP rods produced by die drawing at 55, 57, 60 and 65° C., 30 mm min⁻¹, through a 7 mm die with a 3 mm hex mandrel fitted.

Two 1 cm lengths were cut from the top of each rod and the diameter measured accurately and draw ratio calculated:

$$Draw ratio = \frac{{( Billet diameter )}^{2}}{{( Drawn rod diameter )}^{2}} \times 100$$

One sample of each rod was then shape recovered in hot water (90° C.) and its diameter re-measured when shape recovery was complete. The other sample was placed in water at 37° C., periodically the samples were removed from the water, re-measured then replaced in the water. This was repeated until the diameter remained constant indicating shape recovery was complete. Shape recovery was complete for all of the samples by 30 hours and 25 minutes. The sample diameter data obtained at this time was used to calculate the shape recovery ratio, as described earlier.

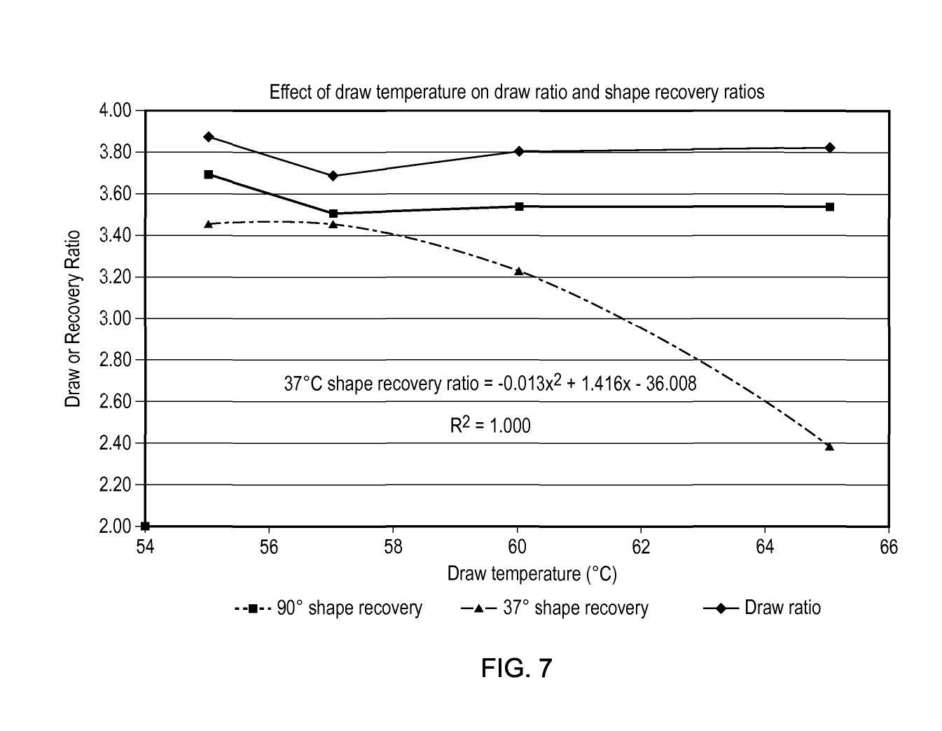

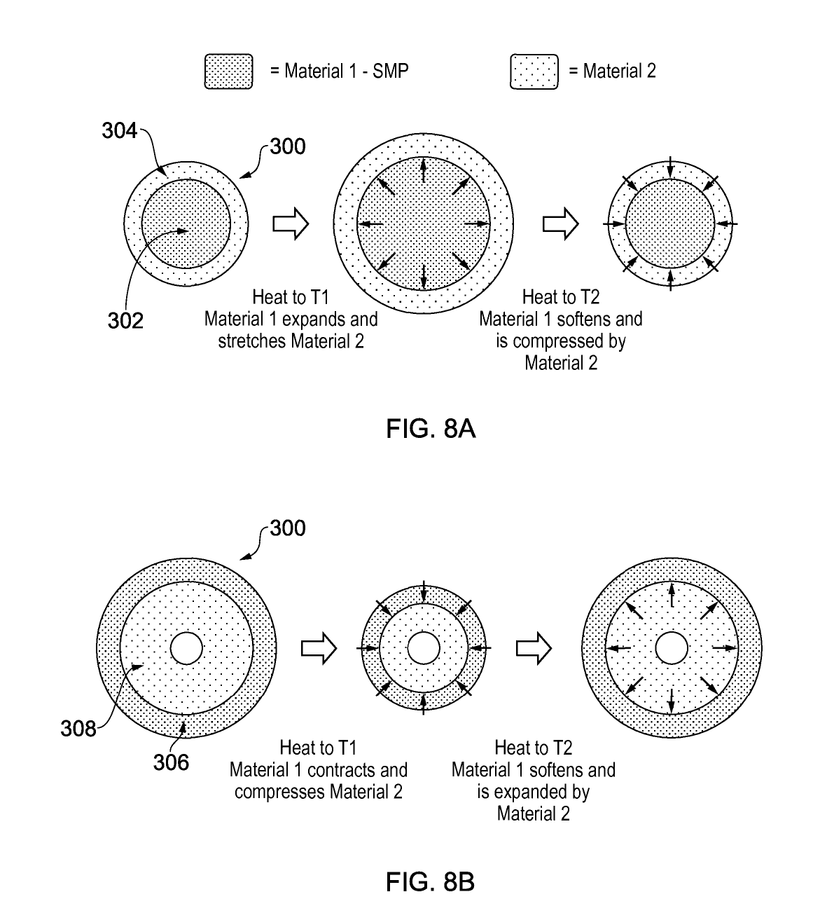

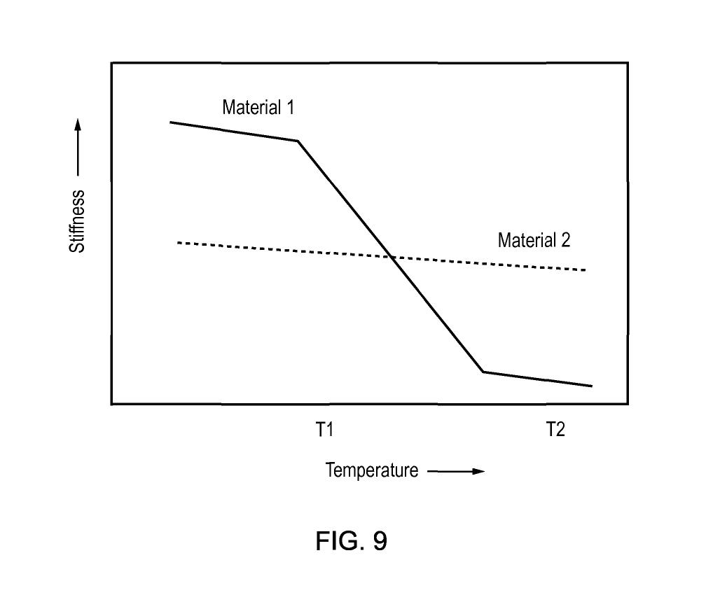

The shape recovery data (FIG. 7) shows that the draw temperature can be used to program the temperature response of the SMP, the recovery ratio being dependent on the temperature the device is exposed to.

1-53. (canceled)

54. A composition for a suture anchor comprising a biocompatible, resorbable Shape Memory Polymer (“SMP”) material which comprises at least one further component, wherein the SMP material is capable of being activated by contact with an aqueous solution and further wherein the device is capable of undergoing a shape change upon said contact with an aqueous solution.

55. The composition according to claim 54, wherein the shape memory polymer (SMP) material is a non-cross linked semi-crystalline shape memory polymer material.

56. The composition according to claim 55, wherein the SMP material comprises a polymer selected from poly (D, L) lactide (PDLA), (lactic-co-glycolic acid) (PDLA), poly (L, co DL) lactide, poly (L-lactide-co-e-captolactone) and copolymers comprising said polymers.

57. The composition according to claim 56, wherein the SMP material comprises an SMP co-polymer.

58. The composition according to claim 56, wherein the SMP material comprises poly (D, L-lactide) at a ratio of between about 60-80% L-lactide and between about 20-40% DL-lactide.

59. The composition according to claim 56, wherein the SMP material comprises poly (D, L-lactide) at a ratio of about 70% L-lactide and about 30% DL-lactide.

60. The composition according to claim 60, wherein the at least one further component is selected from a plasticizer, an inorganic filler, a pharmaceutical agent, a bioactive agent, a magnetic component, and combinations thereof.

61. The composition according to claim 60, wherein the at least one further component is a plasticizer.

62. The composition according to claim 61, wherein the plasticizer is a low molecular weight component selected from the group consisting of DL-lactide, L-lactide, glycolide, e-Caprolactone, N-methyl-2-pyrrolidinone, and a hydrophilic polyol including poly(ethylene) glycol (PEG).

63. The composition according to claim 54, wherein the at least one further component comprises an inorganic filler.

64. The composition according to claim 63, wherein the filler is hydroxylapatite.

65. The composition according to claim 60, wherein the SMP material is activated by contact with an aqueous media having a temperature of approximately 37° C.

66. The composition according to claim 60, wherein the bioactive active agent is selected from a growth factor, an osteogenic factor, an angiogenic factor, an anti-inflammatory agent, and an antimicrobial agent.

67. The composition according to claim 54, wherein the SMP material is activated by heating above its Tg.

68. A device comprising a composition according to claim 54.

69. The device according to claim 68, which comprises a first component comprising the composition comprising the SMP material and a second component, wherein the SMP material has a first activation temperature and further wherein the SMP material is substantially more deformable at a second activation temperature; and further wherein the second component is deformable by the first component at a temperature equal to or greater than the first activation temperature and which is capable of deforming the first component at a temperature equal to or greater than the second activation temperature.

70. The device according to claim 69, which comprises an inner layer and an outer layer, wherein said inner layer comprises the first component and the outer layer comprises the second component.

71. The device according to claim 69, wherein the second component comprises an elastomer.

72. The device according to claim 69, wherein the second component comprises a SMP material having a higher activation temperature than the SMP material of the first component.

73. A method of repairing a soft tissue comprising; placing a device according to claim 69 in bone, passing a flexible member through a soft tissue located adjacent to the bone, tying the flexible member to secure the soft tissue to the body, and activating the SMP material such that the device undergoes a radial expansion in at least a section of its length.

74. A method of treating a bone injury comprising implanting a device according to claim 69 into a bone cavity and activating the SMP material.
